(19) United States
(12) Patent Application Publication
(10) Pub. No.: US 2024/0238055 A1
(43) Pub. Date: Jul. 18, 2024

(54) ROBOTIC SURGICAL INSTRUMENTS WITH DIVERGING FORM FACTORS

(71) Applicant: TITAN MEDICAL INC., Toronto (CA)

(72) Inventors: Jack Alexander Hornsby, Tempsford (GB); Antony R. Burness, Cambridge (GB); Matthew M. Marinovich, London (GB); Mark J. Pawulski, Saffron Walden (GB)

(21) Appl. No.: 18/561,705

(22) PCT Filed: May 26, 2022
(86) PCT No.: PCT/CA2022/050847

§ 371 Date: Nov. 16, 2023

Related U.S. Application Data
(60) Provisional application No. 63/212,921, filed on Jun. 21, 2021.

(51) Int. Cl. A61B 34/30; A61B 34/00

(52) U.S. Cl. CPC A61B 34/30; A61B 34/71; A61B 2034/301

(57) ABSTRACT

A surgical instrument with a divergent form factor includes an elongated shaft assembly having a proximal end portion and a distal end portion. The surgical instrument defines a longitudinal axis and supports a cassette housing on the proximal end portion of the elongated shaft assembly. The cassette housing diverges from the longitudinal axis. The distal end portion of the elongated shaft assembly supports an end effector that is operably coupled to an actuator assembly supported by the cassette housing. The surgical instrument removably couples to a control drive unit of a robotic surgical assembly to enable the actuator assembly to operate the end effector.

### CROSS-REFERENCE TO RELATED APPLICATION

This application is a 371 National Stage Application of International Application No. PCT/CA2022/050847, filed May 26, 2022, which claims the benefit of, and priority to, U.S. Provisional Patent Application No. 63/212,921, filed on Jun. 21, 2021, the entire contents of each of which are hereby incorporated herein by reference.

### TECHNICAL FIELD

This disclosure relates to robotic systems and, more particularly, to instrument form factors for robotic surgical instruments.

### BACKGROUND

Surgical instruments used in laparoscopic and/or robotic surgery generally have a proximal housing and an elongated shaft extending distally from the proximal housing to an end effector. The proximal housing supports actuating mechanisms that may be used to actuate the end effector for performing a surgical task within a body cavity of a patient. Such instruments may be used in applications where there is an area of limited access for an operator. The end effector may be inserted into the area of limited access and the operator may remotely and/or robotically manipulate the instrument via the actuator mechanisms.

### SUMMARY

In accordance with an aspect of this disclosure, a robotic surgical system includes a drive unit and a surgical instrument. The surgical instrument is removably connected to the drive unit and defines a longitudinal axis. The surgical instrument has a proximal end portion with a divergent form factor and a distal end portion supporting an end effector. The surgical instrument further includes an elongated shaft assembly and an instrument cassette assembly. The elongated shaft assembly extends between the proximal and distal end portions of the surgical instrument. The instrument cassette assembly is supported on a proximal portion of the elongated shaft assembly. The instrument cassette assembly includes a cassette housing and an actuator system. The cassette housing is positioned to define the divergent form factor of the surgical instrument. The cassette housing has an inner portion disposed a first distance away from the longitudinal axis of the surgical instrument and an outer portion disposed a second distance away from the longitudinal axis of the surgical instrument. The second distance is farther from the longitudinal axis than the first distance. The actuator system is supported in the cassette housing and operably coupled to the end effector for operating the end effector.

In aspects, the cassette housing may support a transition block assembly that enables at least one cable from the actuator system to extend from the actuator system into the elongated shaft assembly for operably coupling to the end effector. The transition block assembly may couple the cassette housing to the proximal portion of the elongated shaft assembly. The transition block assembly may position the cassette housing at an angle relative to the elongated shaft assembly. The transition block assembly may include a tubular portion that connects the transition block assembly to the elongated shaft assembly. The tubular portion may include a first end portion coupled to the elongated shaft assembly and a second end portion coupled to the cassette housing. The tubular portion may further include a curved portion that curves the tubular portion away from the longitudinal axis and connects the first and second end portions together.

In aspects, the elongated shaft assembly may include a proximal end portion that is disposed at an angle relative to a distal end portion of the elongated shaft assembly to position the cassette housing at an angle relative to the longitudinal axis of the surgical instrument.

In aspects, the actuator system may include a plurality of cable actuator assemblies that connects to the end effector. The actuator system may include a rotation actuator assembly positioned adjacent to the plurality of cable actuator assemblies. The actuator system may include an axial actuator assembly positioned in alignment with the rotation actuator assembly.

According to one aspect, this disclosure is directed to a surgical instrument with a divergent form factor for connection to a robotic surgical system. The surgical instrument includes an elongated shaft assembly, an end effector, and an instrument cassette assembly. The elongated shaft assembly defines a longitudinal axis and has a proximal end portion and a distal end portion. The end effector is supported on the distal end portion of the elongated shaft assembly. The instrument cassette assembly is supported on the proximal end portion of the elongated shaft assembly. The instrument cassette assembly includes a cassette housing and an actuator system. The cassette housing has an inner portion disposed a first distance away from the longitudinal axis of the elongated shaft assembly and an outer portion disposed a second distance away from the longitudinal axis of the elongated shaft assembly. The second distance is farther from the longitudinal axis of the elongated shaft assembly than the first distance. The actuator system is supported in the cassette housing and operably coupled to the end effector for operating the end effector.

In aspects, the instrument cassette assembly may have a rounded or oval configuration.

According to yet another aspect, this disclosure is directed to a surgical system. The surgical system includes a drive unit and a plurality of surgical instruments. Each surgical instrument of the plurality of surgical instruments is removably connected to the drive unit at spaced-apart locations around the drive unit to enable the drive unit to simultaneously operate each surgical instrument. Each surgical instrument defines a longitudinal axis and has a proximal end portion with a divergent form factor and a distal end portion supporting an end effector. The divergent form factors of the plurality of surgical instruments are spaced from one another and define a central passage that extends between the plurality of surgical instruments.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate aspects of this disclosure and, together with a general description of this disclosure given above, and the detailed description given below, explain the principles of this disclosure, wherein:

FIG. 1 is a perspective view of a robotic surgical system being used for a surgical procedure on a patient in accordance with the principles of this disclosure;

FIGS. 2-4 are progressive views illustrating surgical instruments of the robotic surgical system of FIG. 1 being manipulated within a body cavity of the patient;

FIG. 5 is a top view of surgical instruments of a surgical system of the robotic surgical system of FIG. 1, the surgical instruments including a form factor with an angled wire transition and a fanned drive assembly;

FIG. 6 is a perspective view of a proximal portion of one of the surgical instruments of FIG. 5 with a portion thereof shown in phantom for clarity;

FIGS. 7 and 8 are side and top views of FIG. 6, respectively;

FIG. 9 is a top view of other surgical instruments of a surgical system of the robotic surgical system of FIG. 1, the surgical instruments including a form factor with an angled wire transition, a 2×2 cable drive assembly, and axial and rotational drive assemblies that are vertically aligned;

FIG. 10 is a perspective view of a proximal portion of one of the surgical instruments of FIG. 9 with a portion thereof shown in phantom for clarity;

FIGS. 11 and 12 are side and top views of FIG. 10, respectively;

FIG. 13 is a top view of still other surgical instruments of a surgical system of the robotic surgical system of FIG. 1, the surgical instruments including a form factor with an angled wire transition, a 2×2 cable drive assembly, and axial and rotational drive assemblies that are horizontally aligned;

FIG. 14 is a perspective view of a proximal portion of one of the surgical instruments of FIG. 13 with a portion thereof shown in phantom for clarity;

FIGS. 15 and 16 are side and top views of FIG. 14, respectively;

FIG. 17 is top view of further surgical instruments of a surgical system of the robotic surgical system of FIG. 1, the surgical instruments including a form factor with an angled instrument shaft and a fanned drive assembly;

FIG. 18 is a perspective view of a proximal portion of one of the surgical instruments of FIG. 17 with a portion thereof shown in phantom for clarity;

FIGS. 19 and 20 are side and top views of FIG. 14, respectively;

FIG. 21 is a top view of other surgical instruments of a surgical system of the robotic surgical system of FIG. 1, the surgical instruments including a form factor with an angled instrument shaft, a 2×2 cable drive assembly, and axial and rotational drive assemblies that are vertically aligned;

FIG. 22 is a perspective view of a proximal portion of one of the surgical instruments of FIG. 21 with a portion thereof shown in phantom for clarity;

FIGS. 23 and 24 are side and top views of FIG. 22, respectively;

FIG. 25 is a top view of other surgical instruments of a surgical system of the robotic surgical system of FIG. 1, the surgical instruments including a form factor with an angled instrument shaft, a 2×2 cable drive assembly, and axial and rotational drive assemblies that are horizontally aligned;

FIG. 26 is a perspective view of a proximal portion of one of the surgical instruments of FIG. 25 with a portion thereof shown in phantom for clarity;

FIGS. 27 and 28 are side and top views of FIG. 26, respectively;

FIGS. 29A-33A are top views of various surgical instruments of the robotic surgical system of FIG. 1, the surgical instruments each being shown with angled instrument shafts, the angle of each angled instrument shaft progressively increasing from FIG. 29A to FIG. 33A;

FIGS. 29B-33B are side views of respective FIGS. 29A-33A shown relative to a longitudinal axis of the respective angled instrument shafts;

FIGS. 29C-33C are top views of surgical systems of the various respective surgical instruments of FIGS. 29A-33A, the surgical systems being shown with one of the respective surgical instruments of the respective surgical systems removed;

FIGS. 34A-40A are top views illustrating various configurations of surgical instruments of the robotic surgical system of FIG. 1;

FIGS. 34B-40B are side views of respective FIGS. 34A-40A; and

FIGS. 34C-40C are top views of surgical systems of the various respective surgical instruments of FIGS. 34A-40A, the surgical systems being shown with one of the respective surgical instruments of the respective surgical systems removed.

### DETAILED DESCRIPTION

Aspects of this disclosure are described in detail with reference to the drawings, in which like reference numerals designate identical or corresponding elements in each of the several views. As used herein, the term “distal” refers to that portion of structure farther from the user (e.g., clinician), while the term “proximal” refers to that portion of structure, closer to the user. As used herein, the term “clinician” refers to a doctor, nurse, or other care provider and may include support personnel and/or equipment operators. As used herein, the term “cable” refers to one or more wires or fibers that may include metallic or nonmetallic materials, that may have one or more protective casings or insulation (e.g., polymeric material such as rubber or plastic) thereon, and/or that may be twisted together. In some aspects, cables may include one or more nitinol wires.

In the following description, well-known functions or constructions are not described in detail to avoid obscuring the present disclosure in unnecessary detail.

Robotic surgical systems have been used in minimally invasive medical procedures and can include robotic arm assemblies. Such procedures may be referred to as what is commonly referred to as “Telesurgery.” Some robotic arm assemblies include one or more robot arms to which surgical instruments can be coupled. Such surgical instruments include, for example, endoscopes, electrosurgical forceps, cutting instruments, staplers, graspers, electrocautery devices, or any other endoscopic or open surgical devices. Prior to or during use of the robotic surgical system, various surgical instruments can be selected and connected to the robot arms for selectively actuating end effectors of the connected surgical instruments.

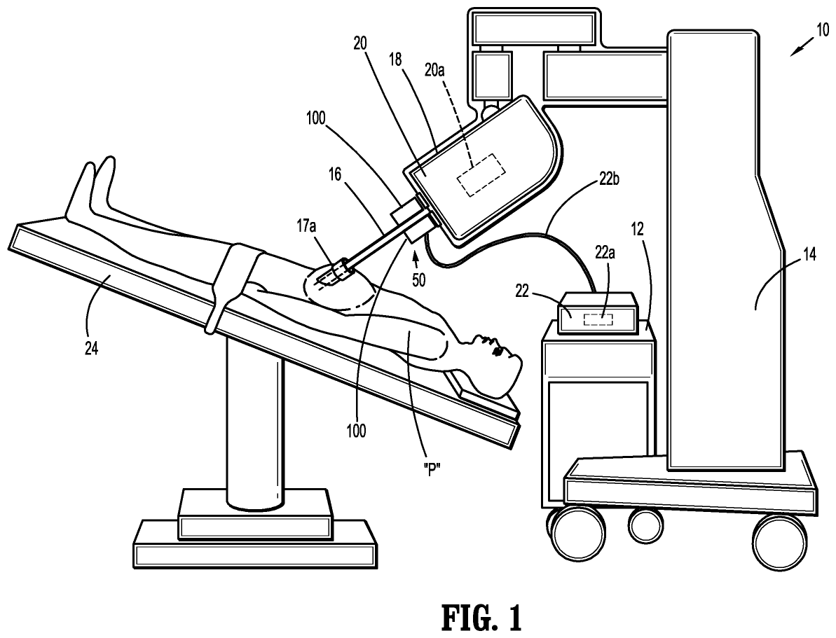

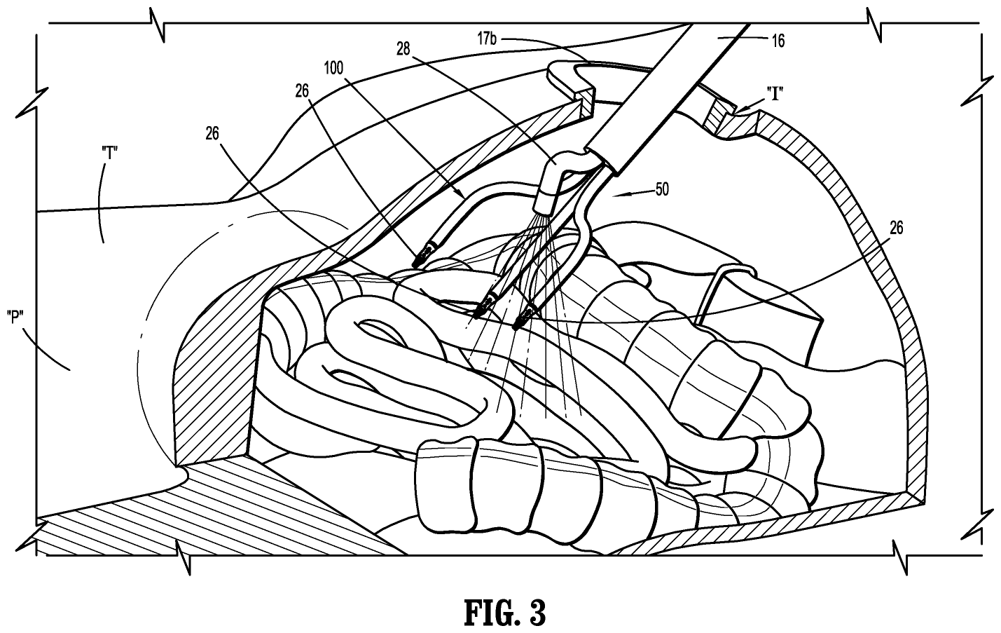

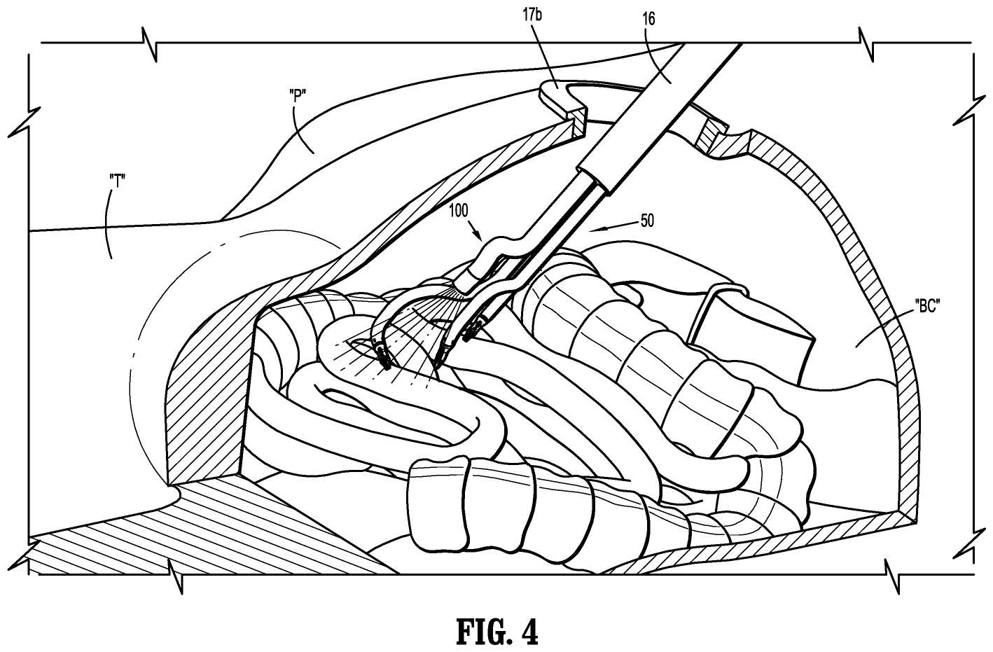

With reference to FIGS. 1-4, a robotic surgical system is shown generally at 10. Robotic surgical system 10 employs various robotic elements to assist the clinician and allow remote operation (or partial remote operation) of surgical instruments 100 of surgical instrument systems 50 of robotic surgical system 10. Various controllers, circuitry, robotic arms, gears, cams, pulleys, electric and mechanical motors, etc. may be employed for this purpose and may be designed with surgical system 10 to assist the clinician during an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

Robotic surgical system 10 includes a workstation 12 and an instrument cart 14. The instrument cart 14 includes one or more surgical instrument systems 50 mounted on a moveable drive unit 18 that houses an instrument drive assembly 20 for manipulating the surgical instrument system 50 and/or independent surgical instruments 100 thereof with the assistance of, for example one or more computing devices or controllers. The surgical instruments 100 can include, for example, graspers or forceps 26, which may be electrosurgical, an endoscope 28, and/or any other suitable instrument that can be driven by one or more associated tool drives (not shown) of instrument drive assembly 20. For example, besides graspers 26 and endoscope 28, the one or more surgical instruments 100 can include dexterous tools, such as needle drivers, staplers, dissectors, cutters, hooks, scissors, coagulators, irrigators, suction devices, etc., or combinations thereof, that are used for performing a surgical procedure.

Surgical instrument system 50 includes an insertion tube 16 defining a plurality of separate conduits, channels or lumens 16a therethrough that are configured to receive, for instance, surgical instruments 100 for accessing a body cavity “BC” of a patient “P.” In other aspects, the insertion tube 16 may define a single conduit, channel or lumen therethrough that is configured to receive, for instance, the surgical instruments 100 for accessing a body cavity “BC” of a patient “P.” In particular, the insertion tube 16 can be inserted through an incision “I” and/or access device 17a, 17b (e.g., a surgical portal, which may include or more seals to facilitate sealed insertion through tissue “T” of the patient “P”) and into the body cavity “BC” of the patient “P”. With insertion tube 16 positioned in the patient “P,” the surgical instruments 100 can be advanced through insertion tube 16 into the body cavity “BC” of the patient “P.” Further, the workstation 12 includes an input device 22 for use by a clinician for controlling the insertion tube 16 and the surgical instrument system 50 and surgical instruments 100 thereof via the instrument drive assembly 20 to perform surgical operations on the patient “P” while the patient “P” is supported on a surgical table 24, for example. Input device 22 is configured to receive input from the clinician and produces input signals. Input device 22 may also be configured to generate feedback to the clinician. The feedback can be visual, auditory, haptic, or the like.

The workstation 12 can further include computing devices and/or controllers such as a master processor circuit 22a in communication with the input device 22 for receiving the input signals and generating control signals for controlling the robotic surgical system 10, which can be transmitted to the instrument cart 14 via an interface cable 22b. In some cases, transmission can be wireless and interface cable 22b may not be present. The input device 22 can include right and left-hand controls (not shown) and/or foot pedals (not shown), which are moved/operated to produce input signals at the input device 22 and/or to control robotic surgical system 10. The instrument cart 14 can include a slave processor circuit 20a that receives and the control signals from the master processor circuit 22a and produces slave control signals operable to control the surgical instrument system 50 during a surgical procedure. The workstation 12 can also include a user interface, such as a display (not shown) in communication with the master processor circuit 22a for displaying information (such as, body cavity images) for a region or site of interest (for example, a surgical site, a body cavity, or the like) and other information to a clinician. While both master and slave processor circuits are illustrated, in other aspects, a single processor circuit may be used to perform both master and slave functions.

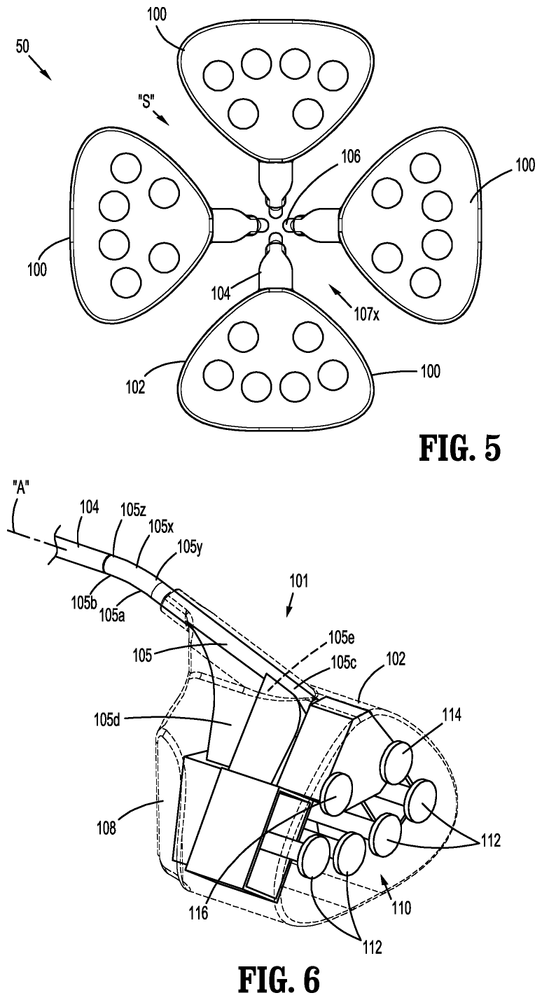

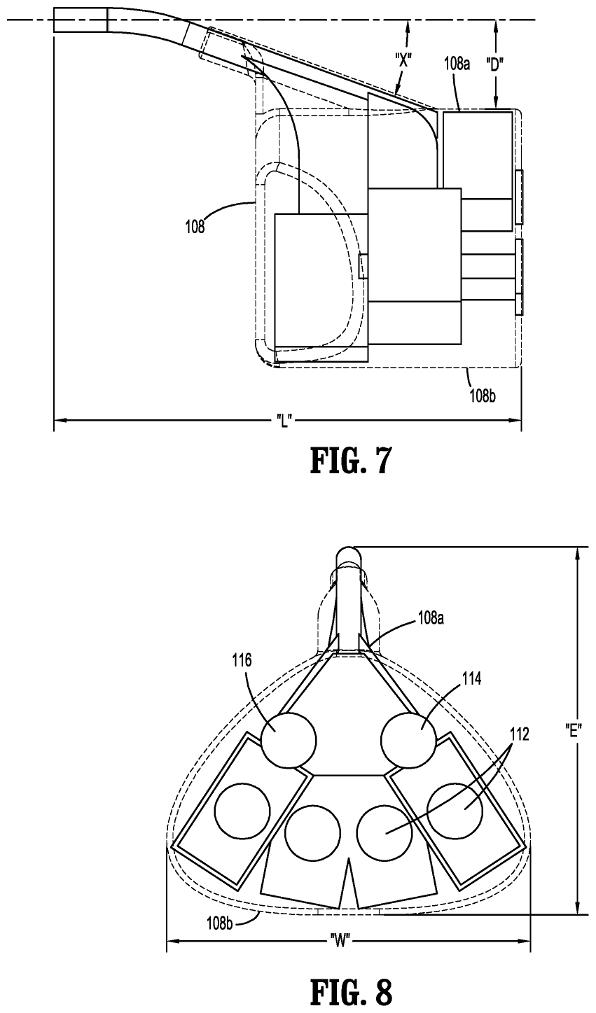

With reference to FIGS. 5-8, surgical instrument system 50 of robotic surgical system 10 can include a plurality of surgical instruments 100. Each surgical instrument 50 is selectively attachable and selectively removable from moveable drive unit 18 (FIG. 1). Although only four surgical instruments 100 are shown, surgical instrument system 50 can include any number and/or type of surgical instruments.

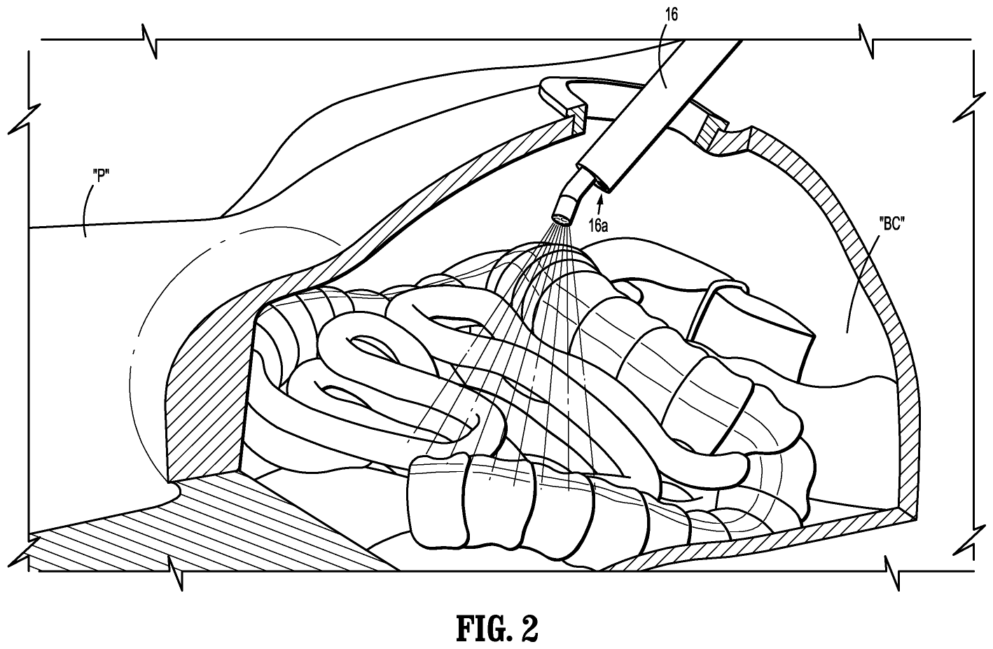

Each surgical instrument 100 of surgical instrument system 50 defines a longitudinal axis “A” and includes an instrument cassette assembly 102 on a proximal end portion thereof, an elongated shaft assembly 104 that extends distally from instrument cassette assembly 102, and an end effector 106 supported on a distal end portion of elongated shaft assembly 104. End effector 106 may include portions (e.g., distal portions) of elongated shaft assembly 104 such as a dexterous wrist assembly that connects elongated shaft assembly 104 to jaw members of end effector 106 and facilitates movement of end effector 106 (e.g., upon a manipulation of one or more cables of an actuator assembly of the surgical instrument 100). End effector 106 is actuatable by instrument cassette assembly 102 for effectuating a surgical procedure. Indeed, actuating end effector 106 can cause end effector 106 to, for example, articulate, pivot, clamp, rotate, etc. relative to the longitudinal axis “A” of surgical instrument 100 for repositioning end effector 106 and/or for treating tissue “T” of the patient “P” as noted above (see FIGS. 2-4).

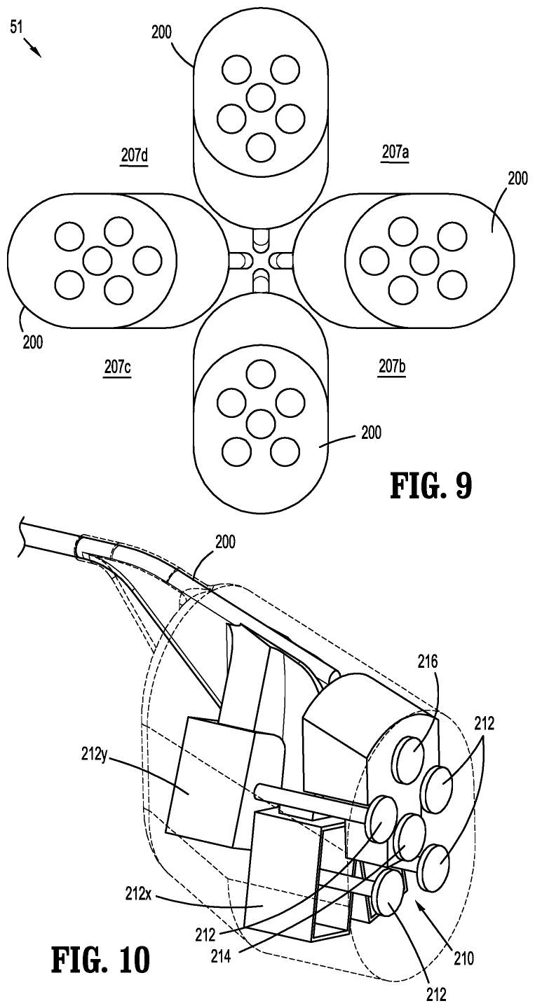

Surgical instrument 100 has a diverging form factor 101 in which instrument cassette assembly 102 is angled offset from longitudinal axis “A” via an angle “X” that may be any suitable angle. For instance, an inner side 108a of a distal end portion of a cassette housing 108 of instrument cassette assembly 102, which may be parallel to longitudinal axis “A,” may be separated from longitudinal axis “A” by a first separation distance “D.” First separation distance “D” may be, for example, 30 mm. An outer side 108b of cassette housing 108 can be separated from longitudinal axis “A” by a second separation distance “E,” which may be, for example 120 mm. A length of form factor 101 may be 215 mm from a proximal end portion of elongated shaft assembly 104. This angled offset provides surgical instrument system 50 with increased space “S” between proximal portions of surgical instruments 100 that facilitate attachment of surgical instruments 100 to movable drive housing unit 18, increased internal component spacing within movable drive housing 18, increased visualization and access points through one or more passages 107x defined between and along a length of surgical instruments 100 (e.g., a central passage 207x and corner quadrant passages 207a, 207b, 207c, 207d defined by areas adjacent pairs of the surgical instruments as shown in FIG. 9), and an improved weight distribution of surgical instruments 100 relative to movable drive housing unit 18. Further still, such offset also advantageously allows for better positioning of Z-axis drive unit bearing shafts to decrease machine mass and inertia, and improves stiffness of the drive unit. Moreover, this feature improves the functional area for a sterile adapter to attach to the movable drive unit so that the sterile adapter can help secure the surgical instruments to movable drive unit. Additionally, by positioning surgical instruments 100 radially apart from one another, the moveable drive unit can be configured to enable common elements thereof to be centrally located. Even further, such spacing facilitates drape management, for example, improved sizing, positioning, and/or configurations of drapes that can be effectively utilized with the disclosed robotic surgical systems. Notably, a width “W” of cassette housing 108 may be, for example, 120 mm.

Moreover, one benefit the extra spacing allows for is a (angled) rack and pinion style mechanism for end effector rotational degree of freedom. This style of mechanism would not work without spacing between instruments and is also very well suited for use with an angled shaft. Having the spacing allows for a longer rack and therefore a higher amount of rotation. The rotation may be achieved with a few other mechanisms (e.g., angled rack and idler gear, cable drum drive, bevel gears, etc.), but this mechanism is by far the most suitable and easiest to implement.

Further, the spacing advantageously improves heat dissipation in the drive unit, provides increased clearance for the drape (such that the drape does not interfere with instruments as instruments move independently in a longitudinal direction), provides increased hand access for facilitating loading and unloading of instruments, and provides increased access to electrical connectors.

Cassette housing 108 of instrument cassette assembly 102 supports an actuator system 110 and may be coupled to elongated shaft assembly 104 by a transition block assembly 105. Transition block assembly 105 may have one or more bends therein to facilitate cable routing therethrough and to help reduce load distribution (e.g., frictional forces acting on such cables as cables translate therealong). Briefly, as seen in FIG. 6, such bends may be disposed at spaced apart locations, for instance, a first bend 105z may be about 15 degrees and a second bend 105y, which is axially offset from first bend 105z, may be about 75 degrees, although any angular range (e.g., between about one degree to about ninety degrees) may be provided for any of the bends in any suitable arrangement.

In certain surgical instruments, cassette housing 108 may be devoid of transition block assembly 105 such that cassette housing 108 directly couples to elongated shaft assembly 104. In one such instance, such surgical instrument may be an endoscope.

With continued reference to FIGS. 5-8, actuator system 110 includes a plurality of cable actuator assemblies 112 for operating end effector 106, a rotation actuator assembly 114 for imparting rotational force to end effector 106, and an axial actuator assembly 116 for imparting axial force to end effector 106. Cable actuator assemblies 112, rotation actuator assembly 114, and axial actuator assembly 116 are arranged in a fanned or oval configuration. Transition block assembly 105 positions instrument cassette assembly 102 with the angled offset and includes a tubular portion 105a with a first end portion 105b coupled to elongated shaft assembly 104 and second end portion 105c coupled to cassette housing 108. Tubular portion 105 further includes a curved portion 105x that connects first and second end portions 105b, 105c. Transition block assembly 105 further includes a transition block 105d that extends from tubular portion 105a. Transition block 105d defines one or more passages or conduits 105e that are disposed in communication with elongated shaft 104 and which are configured to receive electrical, drive, or actuating cables (not explicitly shown) of actuator system 110 therethrough for coupling actuator system 110 to end effector 106.

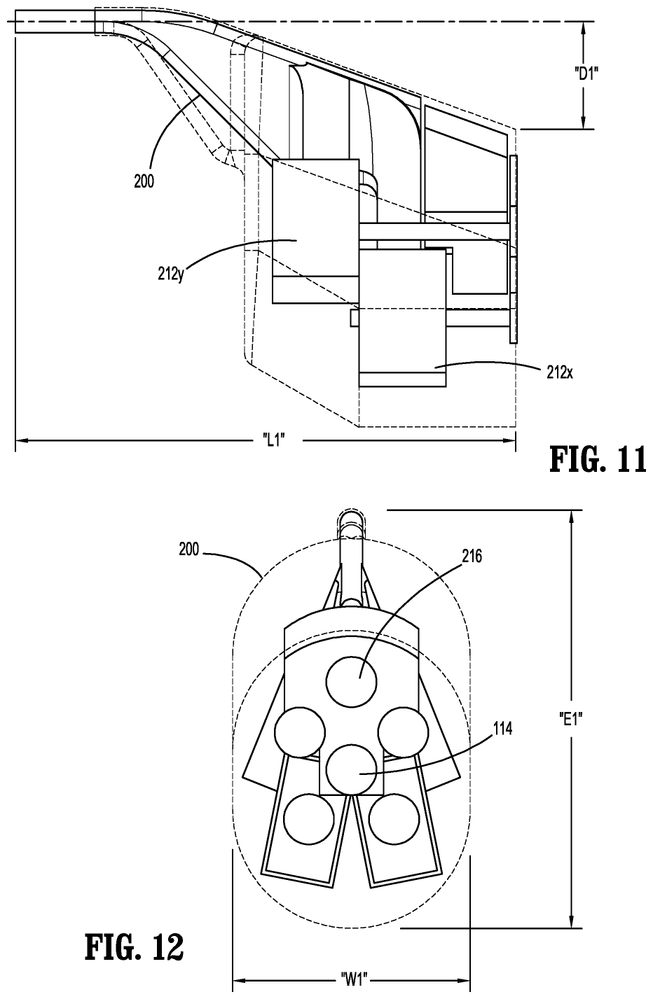

With reference to FIGS. 9-12, another surgical instrument system 51 of robotic surgical system 10 includes surgical instruments 200. Surgical instruments 200 are substantially similar to surgical instruments 100, but include a cassette housing 208 having an oval configuration. Cassette housing 208 supports an actuator system 210 similar to actuator system 110, but actuator system 210 includes cable actuator assemblies 212 disposed in a 2×2 arrangement with a rotation actuator assembly 214 and an axial actuator assembly 216 disposed in a vertical arrangement between cable actuator assemblies 212. As can be seen, for instance, in FIGS. 10 and 11, cable actuator assemblies 212 can include any suitable drive arrangement such as rack and pinion type gearing, which may have an axially offset arrangement (e.g., two proximal racks 212x and two distal racks 212y) helps to elongate cassette housing 208. Advantageously, an elongated cassette housing 208 provides a user friendly form factor with improved ergonomics for facilitating clinician grasp thereof. As can be appreciated, such actuator system 210 can include a pentagonal arrangement of the various actuator assemblies as shown in FIGS. 9, 10, and 12. As seen in FIGS. 11 and 12, first separation distance “D1” may be, for example 40 mm, second separation distance “E1,” may be, for example 150 mm, width “W1,” may be, for example, 85 mm, and length “L1” may be, for example 240 mm.

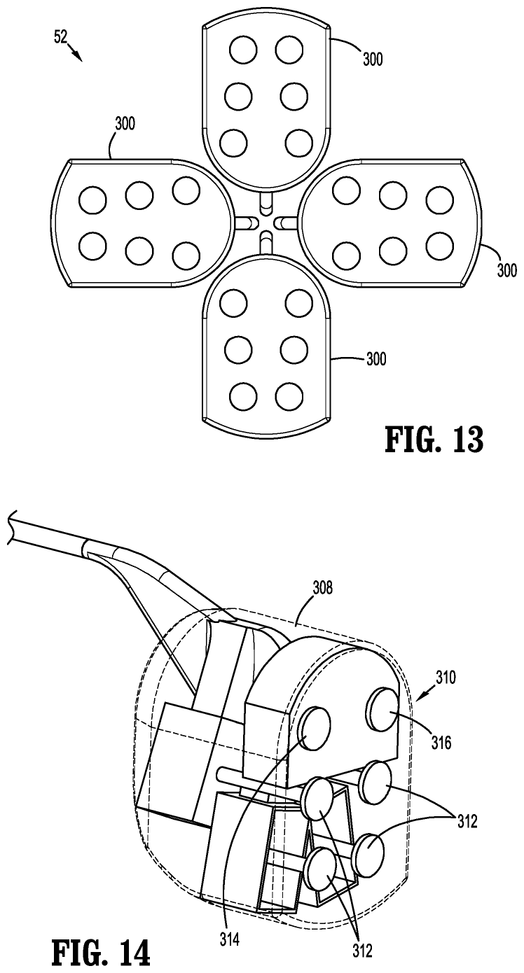

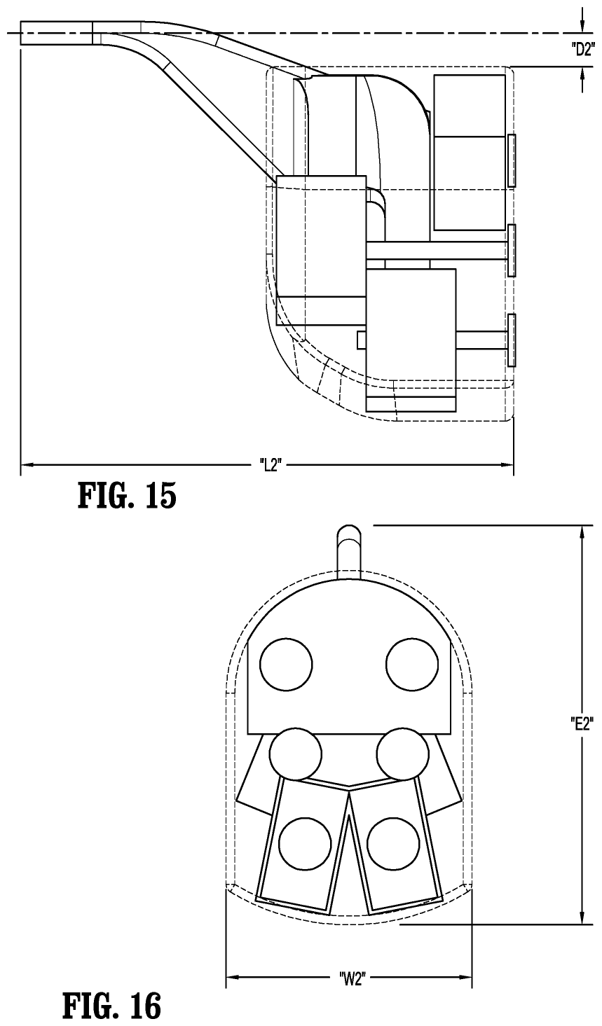

With reference to FIGS. 13-16, yet another surgical instrument system 52 of robotic surgical system 10 includes surgical instruments 300. Surgical instruments 300 are substantially similar to surgical instruments 200, but include a cassette housing 308 having a tombstone configuration. Cassette housing 308 supports an actuator system 310 similar to actuator system 210, but actuator system 310 includes cable actuator assemblies 312 disposed in a 2×2 arrangement with a rotation actuator assembly 314 and an axial actuator assembly 316 disposed in a horizontal arrangement adjacent cable actuator assemblies 312. As can be appreciated, such actuator system 310 can include a tapered arrangement of the various actuator assemblies as shown in FIGS. 13, 14, and 16. As seen in FIGS. 15 and 16, first separation distance “D2” may be, for example 11 mm, second separation distance “E2,” may be, for example 138 mm, width “W2,” may be, for example, 85 mm, and length “L2” may be, for example, 215 mm.

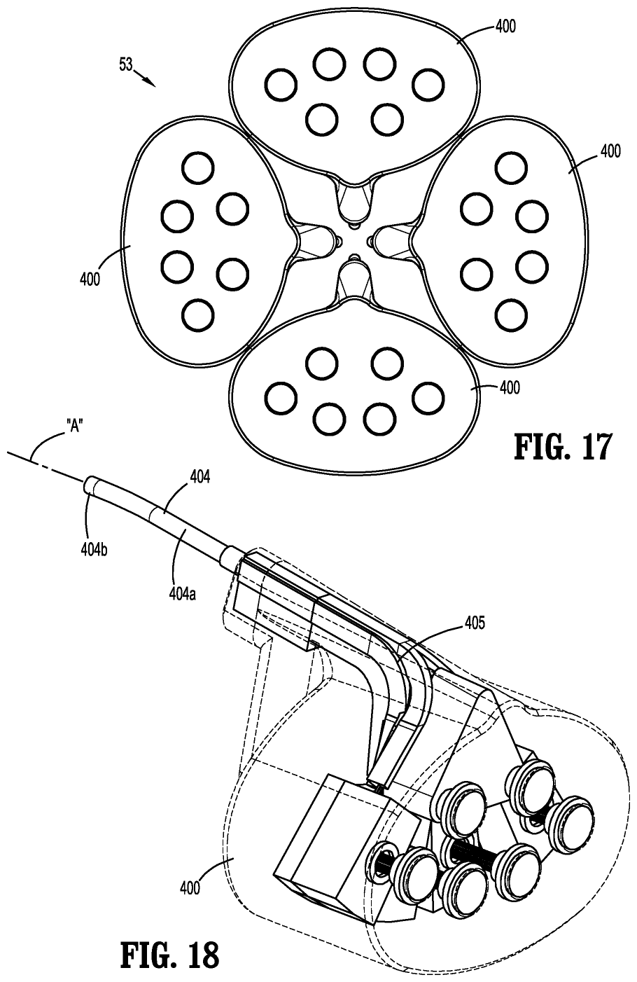

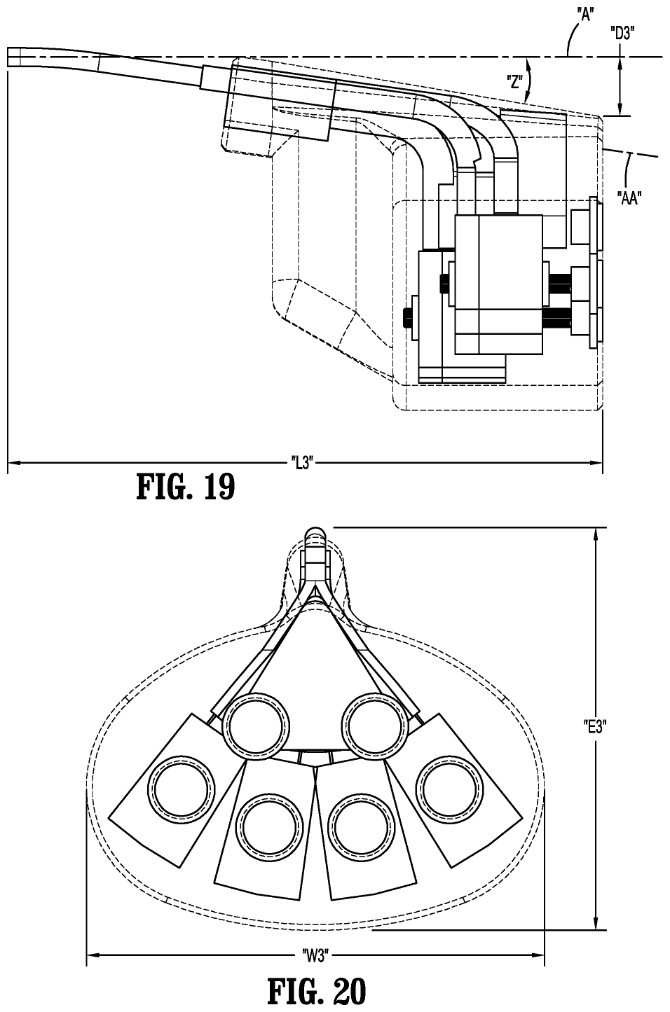

Turning now to FIGS. 17-20, still another surgical instrument system 53 of robotic surgical system 10 is provided, and which is similar to surgical instrument system 50, includes surgical instruments 400. Surgical instrument 400 is similar to surgical instrument 100, but instead of an angled transition block assembly to create a diverging form factor with an angled offset at a proximal end portion of surgical instrument 400, surgical instrument 400 includes a linear transition block assembly 405 and an elongated shaft assembly 404 with an angled proximal end portion 404a that is disposed at an angle “Z” relative to a proximal end portion 404b of elongated shaft assembly 404, and to which a distal end portion of linear transition block assembly 405 connects. Angled proximal end portion 404a defines an angled instrument axis “AA” that is transverse to longitudinal axis “A,” as defined by distal end portion 404b of elongated shaft assembly 404. As seen in FIGS. 19 and 20, first separation distance “D3” may be, for example 22.5 mm, second separation distance “E3,” may be, for example 135 mm, width “W3,” may be, for example, 160 mm, and length “L3” may be, for example, 227 mm.

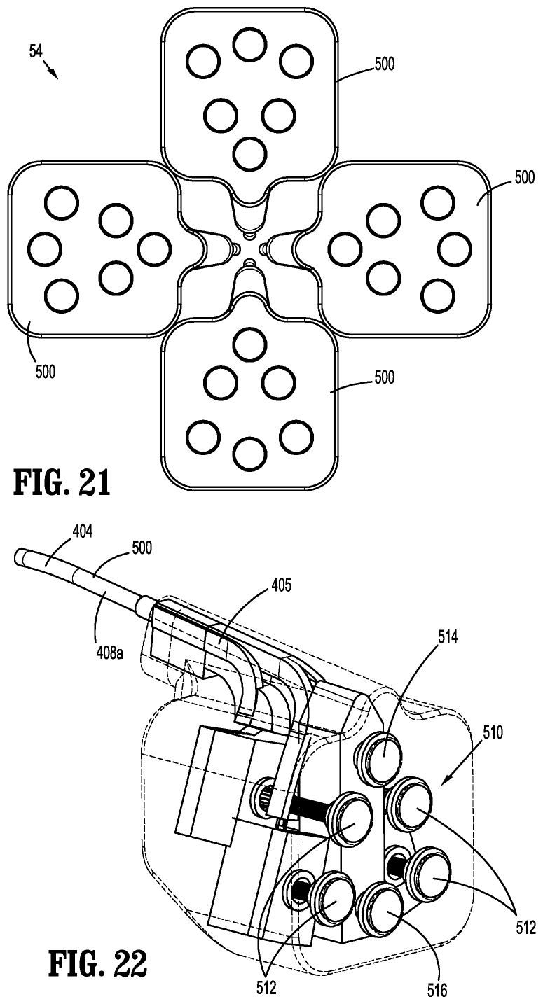

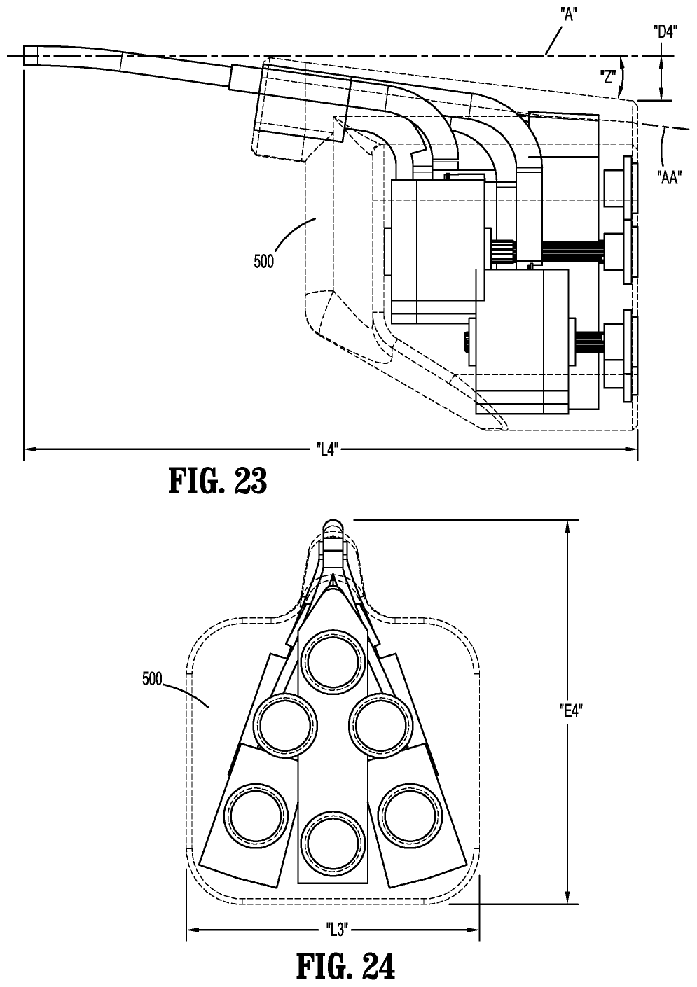

Turning now to FIGS. 21-23, yet another surgical instrument system 54 of robotic surgical system 10 is provided, and which is similar to surgical instrument system 53, includes surgical instruments 500. Surgical instrument 500 is similar to surgical instrument 400 in that it includes a linear transition block assembly 405 and an elongated shaft assembly 404 with an angled proximal end portion 404a that is disposed at an angle “Z” relative to a proximal end portion 404b of elongated shaft assembly 404, and to which a distal end portion of linear transition block assembly 405 connects. Surgical instrument 500 has a squared configuration and includes an actuator system 510 with a 2×2 arrangement of cable actuator assemblies 512, and rotation and axial actuator assemblies 514, 516 disposed in vertical alignment between cable actuator assemblies 512. As can be appreciated, such actuator system 510 can include a triangular arrangement of the various actuator assemblies as shown in FIGS. 21, 22, and 23. As seen in FIGS. 23 and 24, first separation distance “D4” may be, for example 16 mm, second separation distance “E4,” may be, for example 135 mm, width “W4,” may be, for example, 105 mm, and length “L4” may be, for example, 220 mm.

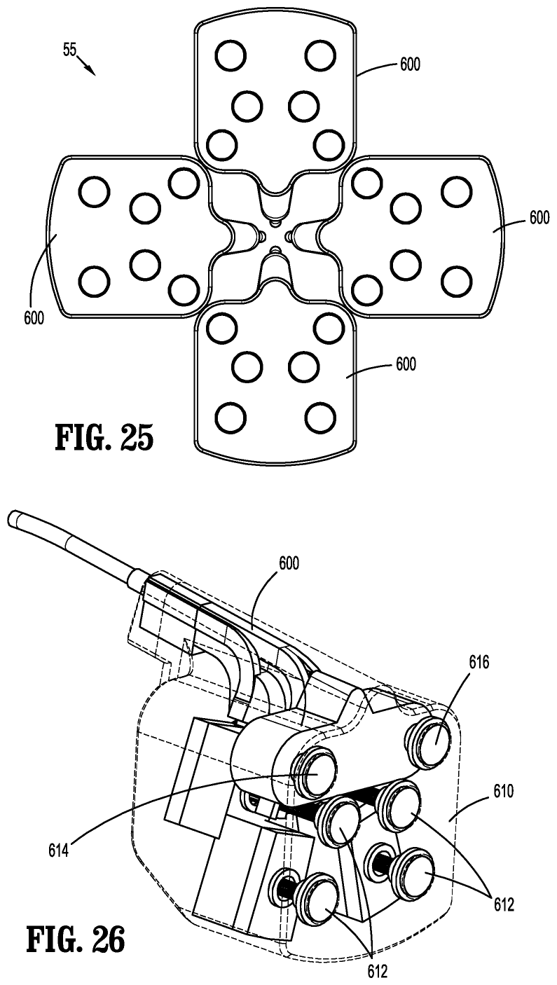

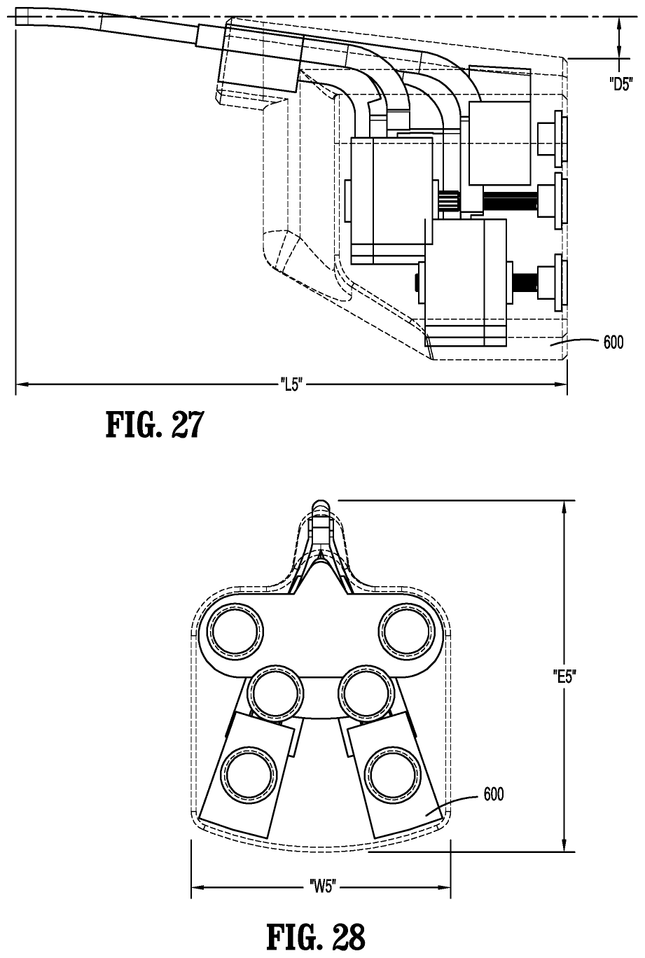

Turning now to FIGS. 25-28, another surgical instrument system 55 of robotic surgical system 10 is provided, and which is similar to surgical instrument system 54, includes surgical instruments 600. Surgical instrument 600 is similar to surgical instrument 500, but includes an actuator system 610 with a 2×2 arrangement of cable actuator assemblies 612, and rotation and axial actuator assemblies 614, 616 disposed in horizontal alignment adjacent cable actuator assemblies 612. As can be appreciated, such actuator system 610 can include an oppositely curved arrangement (e.g., hyperbolic type geometry) of the various actuator assemblies as shown in FIGS. 25, 26, and 27. As seen in FIGS. 27 and 28, first separation distance “D5” may be, for example 17 mm, second separation distance “E5,” may be, for example 130 mm, width “W5,” may be, for example, 105 mm, and length “L5” may be, for example, 225 mm.

FIGS. 29A-33C are progressive views illustrating different angular orientations of angled proximal end portions of elongated shaft assemblies of various surgical instruments to illustrate the differences in spacing between surgical instruments of the respective surgical instrument systems.

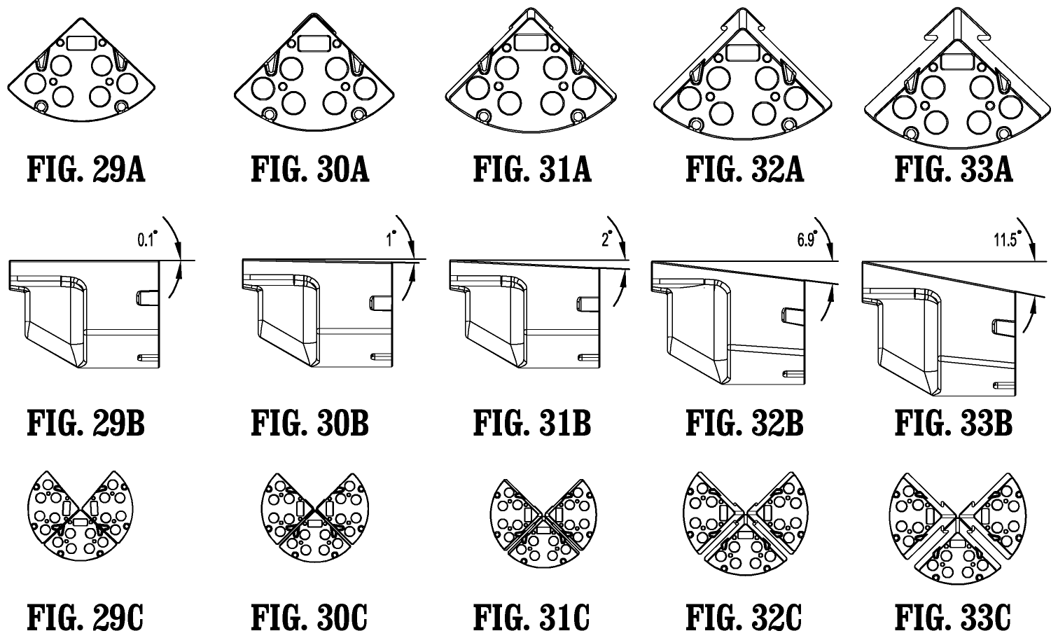

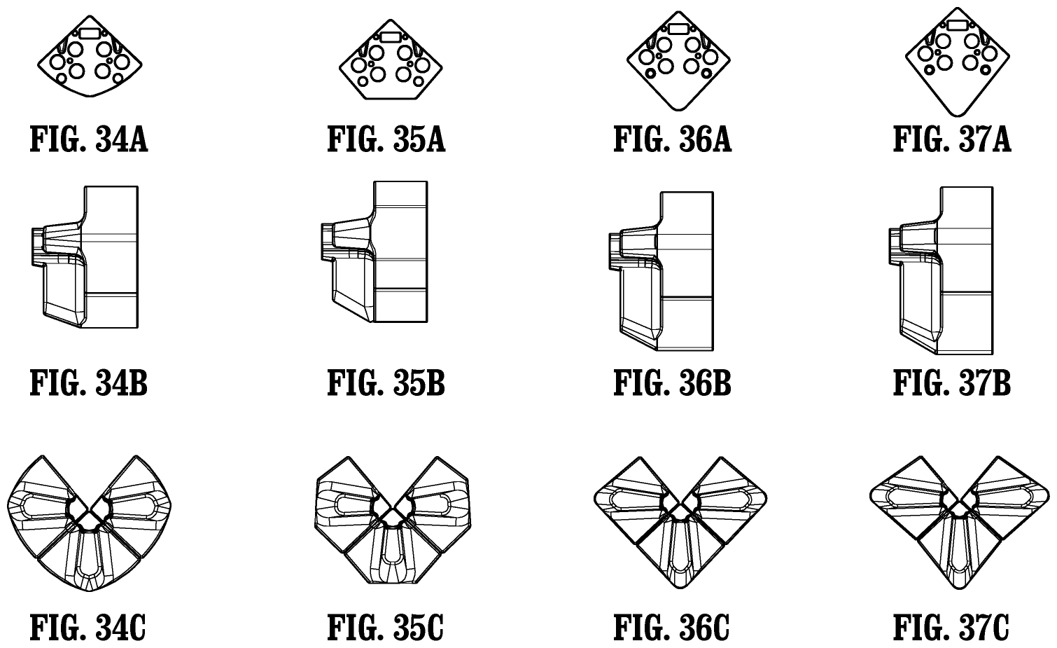

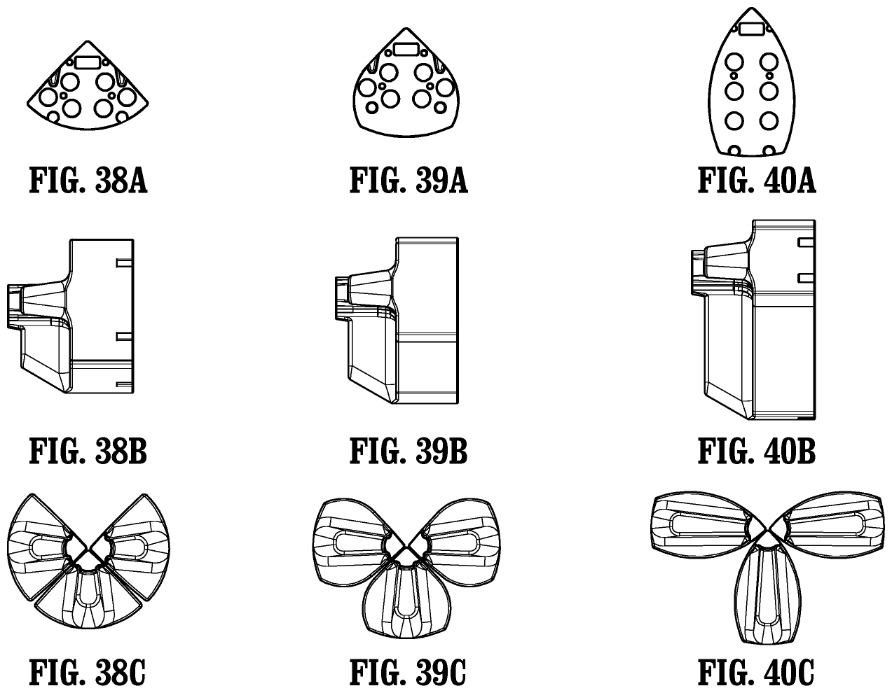

FIGS. 34A-40 are views of various surgical instruments and surgical instrument systems with different configurations. These views are merely examples of various shapes and configurations, but any suitable shape and/or configuration may be provided.

As can be appreciated, the disclosed divergent form factor aspects of the disclosed surgical instruments helps to reduce complexity and friction, helps to improve overall packaging, and helps to limit an amount of motor power and size required for operating these surgical instruments.

Although examples of various dimensions (e.g., distances, lengths, widths, etc.) are described herein, any of these dimensions can be any suitable dimension and/or can be modified as desired for providing any suitable circular, non-circular, and/or polygonal shape or configuration.

The phrases “in an aspect,” “in aspects,” “in various aspects,” “in some aspects,” or “in other aspects” may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

Securement of any of the components of the disclosed devices may be effectuated using known securement techniques such welding, crimping, gluing, fastening, etc.

Persons skilled in the art will understand that the structures and methods specifically described herein and shown in the accompanying figures are non-limiting exemplary aspects, and that the description, disclosure, and figures should be construed merely as exemplary of aspects. It is to be understood, therefore, that this disclosure is not limited to the precise aspects described, and that various other changes and modifications may be effectuated by one skilled in the art without departing from the scope or spirit of the disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

What is claimed is:

1. A robotic surgical system, comprising:
a drive unit; and
a surgical instrument removably connected to the drive unit and defining a longitudinal axis, the surgical instrument having a proximal end portion with a divergent form factor and a distal end portion supporting an end effector, the surgical instrument further including:
an elongated shaft assembly that extends between the proximal and distal end portions of the surgical instrument; and
an instrument cassette assembly supported on a proximal portion of the elongated shaft assembly, the instrument cassette assembly including:
a cassette housing positioned to define the divergent form factor of the surgical instrument, the cassette housing having an inner portion disposed a first distance away from the longitudinal axis of the surgical instrument and an outer portion disposed a second distance away from the longitudinal axis of the surgical instrument, the second distance farther from the longitudinal axis than the first distance; and
an actuator system supported in the cassette housing and operably coupled to the end effector for operating the end effector.

2. The robotic surgical system of claim 1, wherein the cassette housing supports a transition block assembly that enables at least one cable from the actuator system to extend from the actuator system into the elongated shaft assembly for operably coupling to the end effector.

3. The robotic surgical system of claim 2, wherein the transition block assembly couples the cassette housing to the proximal portion of the elongated shaft assembly.

4. The robotic surgical system of claim 3, wherein the transition block assembly positions the cassette housing at an angle relative to the elongated shaft assembly.

5. The robotic surgical system of claim 4, wherein the transition block assembly includes a tubular portion that connects the transition block assembly to the elongated shaft assembly.

6. The robotic surgical system of claim 5, wherein the tubular portion includes a first end portion coupled to the elongated shaft assembly and a second end portion coupled to the cassette housing, the tubular portion further including a curved portion that curves the tubular portion away from the longitudinal axis and connects the first and second end portions together.

7. The robotic surgical system of claim 2, wherein the elongated shaft assembly includes a proximal end portion that is disposed at an angle relative to a distal end portion of the elongated shaft assembly to position the cassette housing at an angle relative to the longitudinal axis of the surgical instrument.

8. The robotic surgical system of claim 1, wherein the actuator system includes a plurality of cable actuator assemblies that connects to the end effector.

9. The robotic surgical system of claim 8, wherein the actuator system includes a rotation actuator assembly positioned adjacent to the plurality of cable actuator assemblies.

10. The robotic surgical system of claim 9, wherein the actuator system includes an axial actuator assembly positioned in alignment with the rotation actuator assembly.

11. A surgical instrument with a divergent form factor for connection to a robotic surgical system, the surgical instrument comprising:
an elongated shaft assembly defining a longitudinal axis and having a proximal end portion and a distal end portion;
an end effector supported on the distal end portion of the elongated shaft assembly; and
an instrument cassette assembly supported on the proximal end portion of the elongated shaft assembly, the instrument cassette assembly including:
a cassette housing having an inner portion disposed a first distance away from the longitudinal axis of the elongated shaft assembly and an outer portion disposed a second distance away from the longitudinal axis of the elongated shaft assembly, the second distance farther from the longitudinal axis of the elongated shaft assembly than the first distance; and
an actuator system supported in the cassette housing and operably coupled to the end effector for operating the end effector.

12. The surgical instrument of claim 11, wherein the cassette housing supports a transition block assembly that enables at least one cable from the actuator system to extend from the actuator system into the elongated shaft assembly for operably coupling to the end effector.

13. The surgical instrument of claim 12, wherein the transition block assembly couples the cassette housing to the proximal end portion of the elongated shaft assembly.

14. The surgical instrument of claim 13, wherein the transition block assembly positions the cassette housing at an angle relative to the elongated shaft assembly.

15. The surgical instrument of claim 14, wherein the transition block assembly includes a tubular portion that connects the transition block assembly to the elongated shaft assembly.

16. The surgical instrument of claim 15, wherein the tubular portion includes a first end portion coupled to the elongated shaft assembly and a second end portion coupled to the cassette housing, the tubular portion further including a curved portion that curves the tubular portion away from the longitudinal axis and connects the first and second end portions together.

17. The surgical instrument of claim 12, wherein the proximal end portion of the elongated shaft assembly is disposed at an angle relative to the distal end portion of the elongated shaft assembly to position the cassette housing at an angle relative to the longitudinal axis of the elongated shaft assembly.

18. The surgical instrument of claim 17, wherein the actuator system includes a plurality of cable actuator assemblies that connects to the end effector.

19. The surgical instrument of claim 18, wherein the actuator system includes a rotation actuator assembly positioned adjacent to the plurality of cable actuator assemblies and an axial actuator assembly positioned in alignment with the rotation actuator assembly.

20. A surgical system, comprising:
a drive unit; and
a plurality of surgical instruments, each surgical instrument of the plurality of surgical instruments removably connected to the drive unit at spaced-apart locations around the drive unit to enable the drive unit to simultaneously operate each surgical instrument, each surgical instrument defining a longitudinal axis and having a proximal end portion with a divergent form factor and a distal end portion supporting an end effector, the divergent form factors of the plurality of surgical instruments spaced from one another and defining a central passage that extends between the plurality of surgical instruments.
